(19) United States
(12) Patent Application Publication
(10) Pub. No.: US 2010/0113874 A1
(43) Pub. Date: May 6, 2010

(54) TELEOPERATED ENDOSCOPIC CAPSULE

(76) Inventors: Marco Quirini, Monsummano Teme (Pistoia) (IT); Robert J. Webster, Nashville, TN (US); Arianna Menciassi, Pontedera (Pisa) (IT); Paolo Dario, Livomo (IT)

Correspondence Address: Steinfl & Bruno, 301 N Lake Ave Ste 810, Pasadena, CA, 91101, US

(21) Appl. No.: 12/594,193

(22) PCT Filed: Apr. 4, 2007
(86) PCT No.: PCT/IT07/00259

§ 371 Date: Jan. 19, 2010

(51) Int. Cl. A61B 1/04

(52) U.S. Cl. 600/109

(57) ABSTRACT

Teleoperated endoscopic capsule for diagnostic and/or therapeutic purposes inside a cavity in a human body, comprising a body (11) having a front part (12) and a rear part (13), locomotion legs (18) able to project from the body (11) and, moving means (19) for said legs (18) housed in the same body (11), a source of energy (16), means for image acquisition (15), means for reception/transmission of signals (17) from and to an operator, for permitting capsule the control and transfer of acquired images. The legs (18) are hinged to the body (11) and are subdivided into two separate groups (20, 21). The moving means (19) comprise two driving devices (22) each one comprising a motor (23) connected to a corresponding worm screw (24) on which a translatable nut screw cursor (25) is engaged. The nut screw cursor (25) is kinematically connected to the legs (18) of a respective rou (20, 21).

### FIELD OF THE INVENTION

The present invention relates generally to the field of endoscopic devices and more precisely, relates to an endoscopic capsule for diagnostic and/or therapeutic purposes, remote controlled, and able to move inside various areas in the human body, and in particular in the gastrointestinal area, more in particular, in a passive manner in the small intestine and in an active manner in the colon.

### DESCRIPTION OF THE PRIOR ART

As is known in recent years there has been an increasing interest in devices that permit endoscopic examination and treatment in the most autonomous and least invasive manner.

For this purpose semi-automatic locomotive solutions have been studied with terminals mounted with a video camera, based on a “inchworm locomotion model, such as the endoscopic device described in WO02/68035. These systems have the drawback of limited possibility of control of the locomotion parameter, and the speed cannot be varied at all. In addition, they also have the drawback that their bodies creep along the walls of the body cavity inside which they move without being able to avoid possible lesions and pathological sites.

Endoscopic devices controlled from the exterior through force fields (a magnetic field for example) have been disclosed; these devices require that the patient wear suitable equipment to generate the field). As an example, the device known as Norika 3 produced by the Japanese company RF System Lab can be taken as a reference. However the use of these devices can create problems and even be dangerous because of possible interference with other biomedical devices applied to the patient. Moreover, this kind of externally controlled endoscopic devices also involve the risk of side effects due to extensive exposure to electromagnetic fields.

A completely autonomous endoscopic device for detecting images with wireless data transmission, integrated in a small capsule, is described in the U.S. Pat. No. 5,604,531. The device comprises a CMOS image generator, a transmitter, LED illumination and energy supply provided by a watch battery. The main limits of this device concern the lack of active locomotion control: the capsule move forward through the effect of normal peristalsis and, during its movement, cannot stopped nor oriented.

Some solutions intended to provide a solution to the problem of the active locomotion control comprise a capsule equipped with autonomous locomotion means controllable by an external operator. In general, these solutions comprise a cylindrical body having a plurality of locomotion modules arranged on the surface that permit the movement inside the body cavity to be explored. Housed in the cylindrical body are a source of energy, a microcontroller to drive the locomotion modules according to commands transmitted through remote control by the operator, a video camera for image acquisition controlled by a micro-controller, a transceiver system to receive the commands transmitted through remote control by the operator and to transmit the acquired images through the video camera.

In any case, these “autonomous” systems require that a gas be introduced to distend the walls of the cavity to be explored so as to permit the movement of the capsule, and an optimised vision of the cavity. The use of this gas is a painful experience for the patient subjected to endoscopic examination.

A solution aimed at avoiding the use of gas is described in WO2006121239 relating to a capsule with integrated locomotion means and a radio control managed by an external operator. The capsule has an external cylindrical body provided with a transparent spherical cap at the end housing a video camera and various electronic means for operation and control as well as for radio signal transmission/reception. The capsule locomotion means are basically composed of hooking teeth that protrude from longitudinal slits formed on the cylindrical body and that are hinged to a block mounted on a nut screw cursor coupled with a worm screw driven by an electric motor present inside, and firmly attached to, the body. When the worm screw is rotated in one direction, the teeth extend outside the body to grip the walls of the cavity to go through and to be examined, and the block with the teeth moves towards the rear end of the body; however, since the teeth are hooked into the cavity walls, this results in a forward movement of the body in the cavity similar to a creeping movement. When the worm screw is turned in the opposite direction, the block performs a forward axial movement, while the teeth are retracted back inside the body and the capsule remains still.

However fully satisfactory the component miniaturisation may be, the capsule in this solution is unable to turn or tilt to observe details of the cavity under examination. Moreover, it is unable to perform a reverse movement nor can it move in vertical direction, in view of the fact that for a part of the locomotion cycle, none of the teeth are able to hook into the cavity wall. Moreover, the capsule is unable to adapt itself to the various geometries featuring the gastrointestinal tract and it does not comprises any systems able to distend the surrounding intestinal walls adequately, an aspect which is very important from a diagnostic viewpoint to permit optimal observation of intestinal tissue.

Another solution aimed at avoiding the use of gas, is disclosed in WO2005082248. In particular, in this case the locomotion modules are formed by six legs hinged to the cylindrical body and controlled by a drive system composed of wires connected to each leg and acting in opposition to displace it angularly around the hinge axis. The wires are connected to electric contacts by transmission means. Both the legs and the wires are made in SMA (Shaped Memory Alloy). One of the two opposite wires is heated through the passage of electrical current, bringing it to the phase transition temperature of the SMA, resulting in the contraction of the wire (the cold wire is deformed through the action of the hot wire) and the rotation of the leg. When the electrical supply is cut off, the temperature is lowered and the wire stops its traction force, permitting the counteracting wire, heated successively, to contract, thus completing the return movement of the leg and at the same time, bringing the first wire back to its original length. The legs project in a radial direction in relation to the axis of the cylindrical body, and in an equally distanced manner around said axis, so that when the cavity tracts to be explored have a cross-section smaller than the size of the capsule with the legs projected, it provokes the expansion of the cavity.

However advantageous this solution may be as regards the possibility of directing the capsule, the compact size and the “distension” of the tracts to be explored compared to the prior art capsule devices, it nevertheless possesses certain aspects to be improved; these aspects are mainly related to the high power consumption due to the wire heating, this resulting in the reduction in the operating range of the device. Moreover, the force generated by the SMA wires can be insufficient for the complete expansion of the intestinal lumen, this resulting in a difficult forward movement due to the friction provided by the non-distended tissue. Furthermore, not always the legs are able to remain attached to, or in adhesion with, the cavity walls because of the wall irregularity and, slippery surface. To ensure adherence, the force of contact must be increased, but this requires the use of wires with a larger section, resulting in a size increase and greater energy consumption, as well as a reduction in the frequency of the locomotion cycle because of the amount of heat to be eliminated.

The main object of the present invention is to provide an endoscopic capsule having autonomous movement and energy supply within the body cavity, with the possibility of controlling the movement from the exterior to permit medical, diagnostic and therapeutic procedures, to be carried out and in particular, to be able to transmit images of the interesting areas in the body cavity through which it passes.

Another object of the present invention is to provide a teleoperated endoscopic capsule able to move in an autonomous manner inside the cavity to be explored without the need for using gas to expand the cavity walls.

Another object of the present invention is to provide a teleoperated endoscopic capsule that can adapt well to the environment in which it is placed without provoking, irritation or injury to the surrounding tissues.

A further object of the invention is to provide an endoscopic capsule equipped with autonomous locomotion means, wherein its movement can be easily stopped, accelerated or reduced according to need through an external remote control.

Another object of the invention is to provide an endoscopic capsule equipped with autonomous locomotion means that is able to turn corners easily.

### SUMMARY OF THE INVENTION

These objects are achieved with a teleoperated endoscopic capsule for diagnostic and/or therapeutic purposes inside cavities in the human body, comprising a body defining a front part and a rear part, locomotion legs able to extend from said body, and moving means for said legs housed within the body, an energy source, means for acquiring images, means for signal reception/transmission from and to an operator in order to permit capsule control and the transfer of the acquired images. The legs are hinged to said body and are divided into two separate groups, the moving means comprising two driving devices, each one comprising a motor connected to a corresponding worm screw on which a translatable nut screw cursor is engaged, said nut screw cursor being kinematically connected to the legs of a respective group of legs.

### BRIEF DESCRIPTION OF THE DRAWINGS

Further characteristics and advantages of the endoscopic capsule according to the present invention will be made apparent from the following description of its embodiment, provided as a non-limiting example with reference to the appended drawings wherein:

FIG. 1 shows an axonometric view of a capsule according to the invention, illustrating the locomotion legs in their most extended position;

FIG. 2 shows an axonometric view of the capsule of FIG. 1, partially in section;

FIG. 3 shows an axonometric view of internal components of the capsule in the previous figures, in particular the device for the movement of the legs;

FIG. 4 shows a front view of the capsule, on a plane perpendicular to the axis of the capsule body;

FIG. 5 shows a schematic side view of part of the device which drives the leg movement, in which the most outspread position of a leg and the least outspread position shown in dotted line are depicted;

FIG. 6 shows a schematic side view of a part of the leg movement driving device, partially in section;

FIG. 7 shows a front view of a part of the driving device.

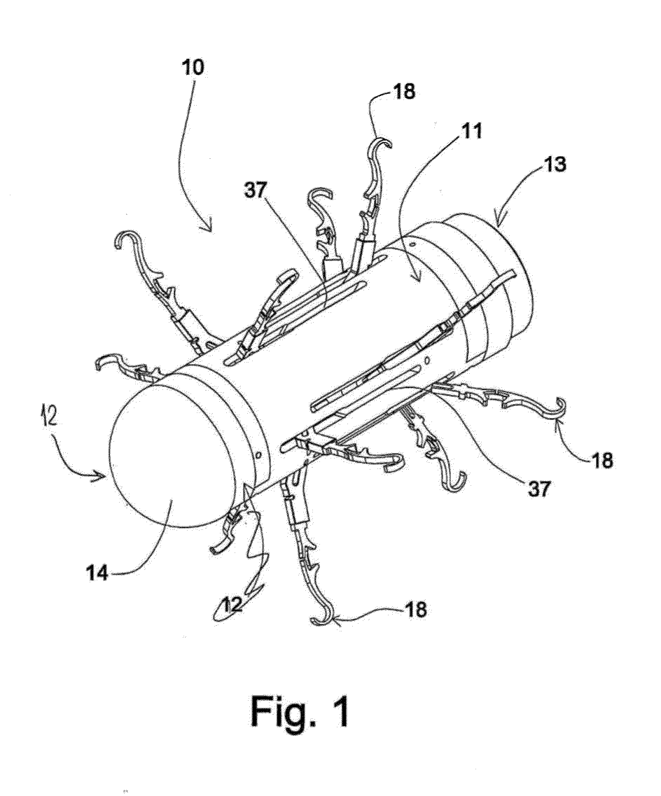

### DETAILED DESCRIPTION OF THE INVENTION

With reference to the aforesaid figures, a teleoperated endoscopic capsule for diagnostic and/or therapeutic purposes according to the invention is identified throughout by the numeral 10.

The capsule 10 comprises a body 11, having a substantially cylindrical shape that defines a front part 12 and a rear part 13.

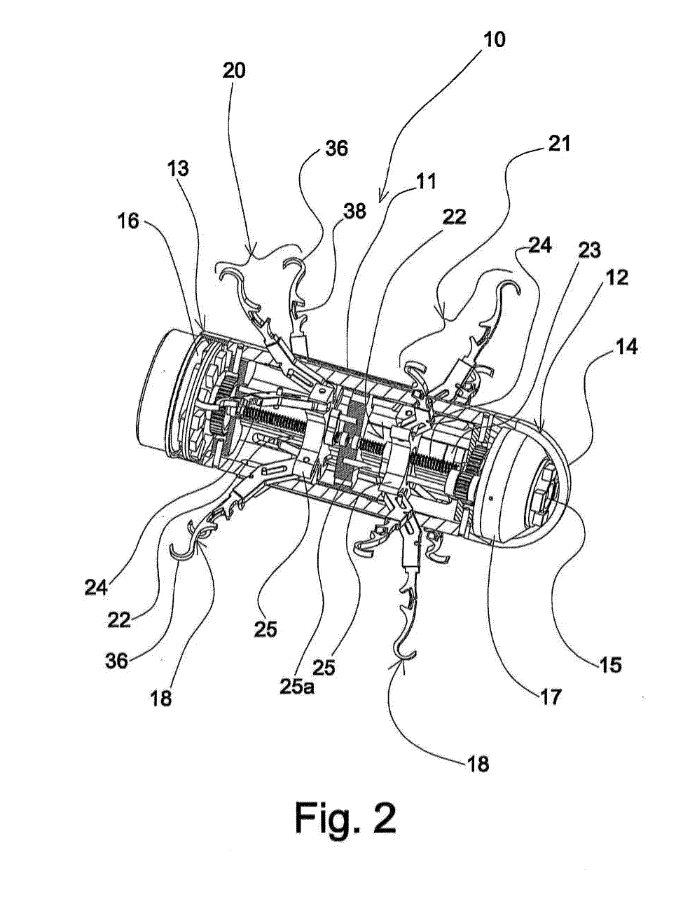

At the end of the front part 12 the body 11 is provided with a transparent dome 14, inside which are arranged means for image acquisition 15, such as a digital video camera (associated lighting means of the known type, not shown) connected to a source of electrical energy 16 such as a watch battery, for example; both the video camera and the battery are shown schematically in FIG. 2.

Means for signal reception/transmission 17 to and from the external operator are housed under the dome 14, to permit remote control of the capsule and the transfer of the acquired images to an external terminal, with which the operator is able to interface. These means are substantially of the known type, such as those described in the international patent application WO2005082248, but other functionally equivalent means can be used in alternative.

It is obvious how, in other embodiments, the video cameras can be more than one in number, such as two for example, positioned in the front part 12 and rear part 13 of body 11 respectively.

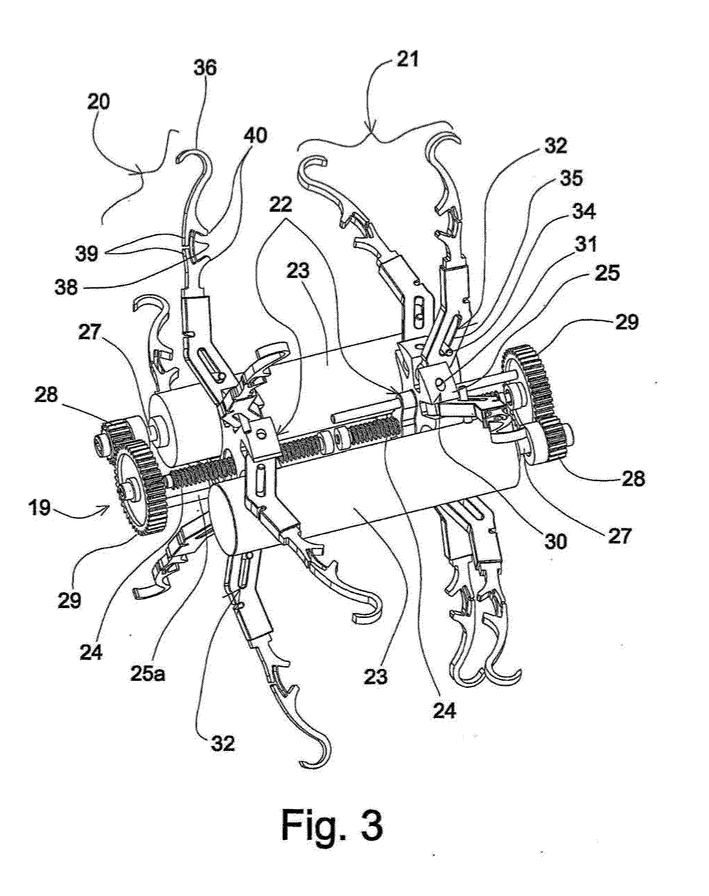

The, capsule 10 also comprises locomotion legs 18 able to project from the body 11; the means 19 for moving the legs 18, illustrated in particular in FIG. 3, are positioned inside body 11. In particular, the legs 18 are divided into two separate groups; a first group 20 is positioned towards the front part 12 the body 11, while a second group 21 is positioned towards the rear part 13.

FIGS. 2 and 3 show how the moving means 19 comprise two separate driving devices 22 for the legs 18, which operate on the first and second groups 20 and 21 respectively; preferably, said driving devices 22 are substantially of the same type, as described below.

Each driving device 22 comprises a motor 23 connected to a corresponding worm screw 24 engaged with a nut screw cursor 25 kinematically connected to the legs 18. The nut screw cursor 25 is forced to translate along the same worm screw 24 because of the slidable coupling to a guide bar 25a, parallel to the worm screw 24 and connected rigidly to body 11.

For example motor 23 is a direct current brushless type electric motor produced by Namiki Precision Jewel Co., Ltd., having an external diameter of 4 mm and a total length of 16.2 mm; said motor has an integrated speed reducer and the maximum delivered torque at the speed reducer shaft is equal to 2.92 mNm (the same motor without the speed reducer has a maximum delivered torque at the shaft equal to 0.058 mNm); said electric motor is powered by the battery 16.

In particular, according to an important characteristic of the invention, the worm screws 24 of the two driving devices 22 are coaxial with the axis of body 11 and the motors 23 are positioned with their drive shafts 27 parallel to the axis of body 11, and therefore on the opposite sides with respect to the common axis of the worm screws 24.

Each motor 23 is connected to the corresponding worm screw 24 by means of a standard gearing with a transmission ratio less than 1; in particular the standard gearing is formed by a pinion 28 fixed at the end of the drive shaft 27 of motor 23 and a gearwheel 29, having a diameter larger than the pinion 28, fixed at the end of the worm screw 24. The transmission ratio ranges between 0.420 and 0.430, preferably equal to 0.425; this is an optimal value in terms of the force delivered to the legs with the space occupied by the standard gearing. The motors 23 are positioned opposite to each other, and therefore the gearwheels 29 of the two worm screws 24 are positioned at each end of the screws.

The moving means 19 of the legs 18 also comprise electronic means (for simplicity not numbered in the figures) substantially of the known type, such as those described in the aforesaid international patent application WO2005082248, able to control the independent motion of the two groups of legs 18, according to the commands from the external controller.

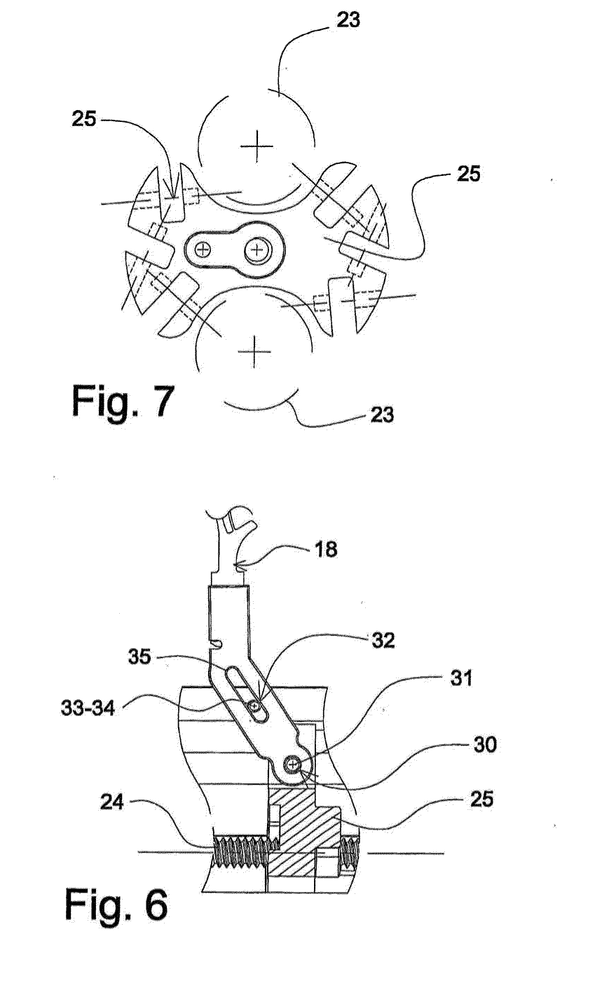

Each group 20 and 21 of legs 18 is connected to a respective nut screw cursor 25 (see in particular FIGS. 5, 6 and 7). Preferably, at a first end of each of its own legs 18, each group 20 and 21 has a first restraint 30 composed of a rotational attaching hinge 31 fixed to the nut screw cursor 25, and at an intermediate position of the same leg 18, a second restraint 32 that allows a roto-translation, with respect to a fixed point 33 of the body 11, the rotation axis being parallel to the axis of the hinge 31.

In particular, said second restraint 32 comprises a rotation pin fixed to the body 11 and a guide groove 35 defined along the corresponding leg 18; said pin 34 and groove 35 are coupled together with relative sliding, to allow the aforesaid rototranslation to be performed.

In practice, each nut screw cursor 25 can translate along the relative worm screw 24 from an initial position, in which the corresponding legs 18 are in their position of minimum extension (as can be seen in the part marked with the dotted line shown in FIG. 5), laid along body 11, to a final position, in which the legs 18 are in their maximum extension, projected towards the exterior of the body 11 with a angle such that the free ends 36 of the legs are positioned at the maximum distance from the axis of the body 11 (the part marked with the dark line in the drawing in FIG. 5).

The driving devices 22 are housed inside the body 11, which is formed with longitudinal slots 37 on the external surface, passing from the interior towards the exterior, to permit the legs 18 to protrude. More in particular, on the body 11 there are provided two groups of angularly equispaced slots, one group for each group of legs, extended respectively from the front end and from the rear end of the body 11 as far as an intermediate position thereof. The slots of one group are not aligned with the corresponding slots of the other group, but are staggered two by two, even though they are positioned closely to one another.

Advantageously, each group of legs 20 and 21 is formed of six legs 18. This choice is the result of a number of tests performed with capsule prototypes presenting different numbers of legs. It was possible to observe from these tests that the locomotion performance increases with an increase in the number of legs. This is due to the fact that with an increased number of legs, the force of propulsion as well as the distension of the intestinal wall is distributed in a much more uniform manner. Therefore, while complying with the dimensional limits imposed by the diameter of the endoscopic capsules available on the market (approximately 11 mm), the number of legs has been maximised as far as twelve in number (six for each group). This number of legs achieves the aims proposed, as will be described more clearly below.

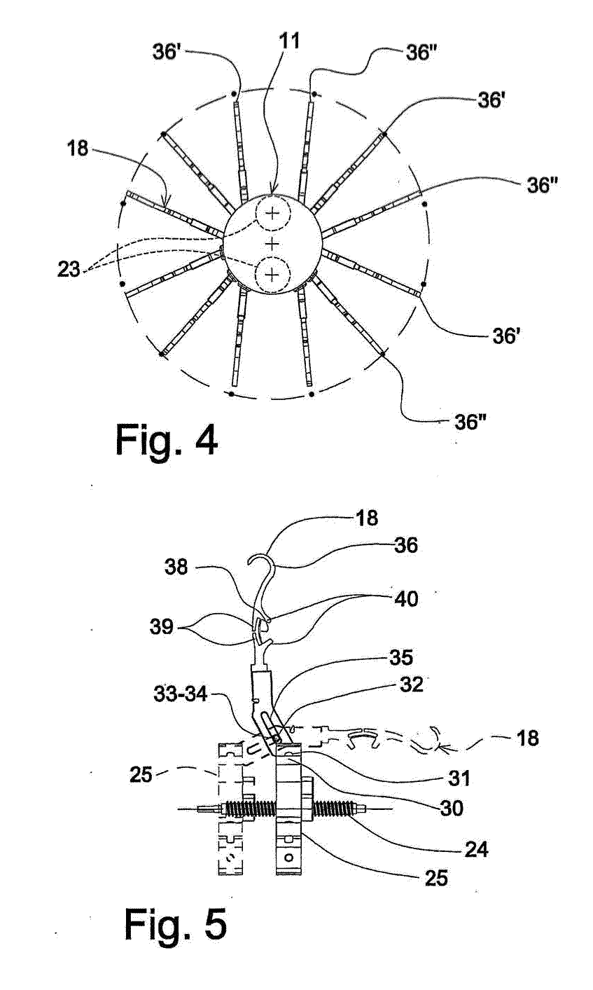

In FIG. 4 the free ends of the legs of the first group 20 are identified with the numeral 36′, while the free ends of the legs of the second group 21 are identified with the numeral 36″. As shown in FIG. 4, the projections of the free ends 36 of the legs 18 of both the first group 20 and the second group 21 on a plane orthogonal to the axis of the corresponding worm screws 24 (coinciding with the axis of body 11) are arranged substantially on a same circumference, shown by the dotted line, whose centre coincides with said axis.

As can be seen from the figure, the free ends 36 are set at substantially the same angular distance from each other along the circumference. In particular, as shown in FIG. 4, when the legs 18 of both groups 20-21 are in their outspread position, the projections of the free ends on the plane orthogonal to the axis of the corresponding worm screw 24, are arranged on a same circumference. More precisely, the legs belonging to the two groups alternate in the projection on the plane orthogonal to the axis of the capsule and, for dimensional reasons, the two legs of each group farthest from the respective motor are slightly displaced on an angle (about 4°) in relation to their ideal position. The above arrangement is required to avoid the degeneration of the volume of the capsule body that accounts for the equal distance spacing of the legs. More precisely, should the longitudinal slots of the aforesaid legs (slots inside which the said legs house when they fold to their completely retracted position) be arranged at the same angular distance in relation to one another, this would result in a collision between the legs during the retraction stage. This interference condition has been avoided by spacing the legs at a slight distance from each other.

In an intermediate point between the free end 36 and the guide groove 35, each leg 18 is formed with an elastic knee portion 38 that forms a further degree of freedom to adapt the leg to the yielding nature of the tissue with which it comes into contact; in other words the terminal portion of the leg is elastically flexible around the knee portion.

Two opposing extensions 39 are positioned near the knee portion 38, to limit the leg 18 rotation for a few degrees in its extension direction, while another pair of extensions 40 can be positioned at the opposite side of the leg 18 to abut each other after a wide rotation around the knee 38. The pair of extensions 40 therefore limits the amount of flexion to which the leg 18 could be subjected, in order to prevent any possible damage thereof.

As shown in the figures, the free end of each leg is substantially hook-shaped for gripping the mucous membrane of the intestine to permit the forward movement; the size of the hooks is smaller than the thickness of the mucous membrane of the intestine (0.2 mm), in this way they do not damage the underlying tissue.

The function of the two groups of legs is different. The second group 21 is mainly aimed at driving the capsule motion, while the first group 20 is mainly aimed at attaching the capsule to the walls of the cavity under exploration, to facilitate the curved trajectories and to distend the walls to provide an optimal view thereof.

The endoscopic capsule according to the invention can be advantageously coated with a biocompatible and biodegradable layer that prevents the accidental outward extension of the legs during ingestion, making the swallowing action easier and safer. When the capsule reaches the stomach, the coating is destroyed by the acidity of the environment thus permitting the leg movements.

This capsule structure makes it possible to respect important constructive and dynamic parameters such as compact size (a length preferably between 24 mm and 28 mm), widespread angles for the legs (preferably between 90° and 130°), internal size that is sufficient to house the electronic components (thanks to motors that do not occupy more than 10.5% of the total volume of the body 11) controlled strength at the free ends of the legs (preferably between 1.8N and 3.2N) and number of legs (between 8 and 12).

Furthermore, thanks to the structure of the capsule according to the invention, it is possible to use a number of legs (twelve, in the example described) that is much larger than in other prior art capsules.

This provides several advantages, among which: a) the possibility of distending the cavity walls more easily for exploration, whereby the use of gas to expand the cavity is avoided; b) the possibility of low interaction force of each leg with the cavity walls, but with an overall force strong enough to permit hook gripping and locomotion along the walls, with the obvious advantage concerning the risk of tissue irritation and damage, and; c) greater flexibility in adjusting the locomotion speed.

The capsule according to the invention can be modified and varied in several ways, all of which are within the scope of the invention; all the details may further be replaced with other technically equivalent elements. In practice, the materials used, so long as they are compatible with the specific use, as well as the dimensions, may be any according to the requirements and the state of the art.

Wherever the characteristics and techniques described in any claim are followed by a specific reference, these have been included as an example for the sole purpose of making the claim descriptions easier to understand, and therefore they impose no limits on the interpretation of the element they refer to.

1. A teleoperated endoscopic capsule for diagnostic and/or therapeutic purposes inside a cavity in a human body, comprising
a body having a front part and a rear part,
locomotion legs able to project from said body and,
moving means for said legs housed within the body,
a power source,
means for image acquisition, and
means for reception/transmission of signals from and to an operator for permitting control of the capsule and transfer of acquired images,
wherein
said legs are hinged to said body and are subdivided into two separate groups,
said moving means comprise two driving devices each one comprising a motor connected to a corresponding worm screw on which a translatable nut screw cursor is engaged, and
said nut screw is kinematically connected to the legs of a respective group.

2. The teleoperated endoscopic capsule according to claim 1, wherein said at least one first group comprises, at a first end of each leg, a first restraint formed by a hinge, for pivotally fixing the leg to the relevant nut screw cursor and, in an intermediate position of the same leg, a second restraint for permitting a roto-translation, with the rotation axis parallel to the axis of said hinge, with respect to a fixed point of said body.

3. The teleoperated endoscopic capsule according to claim 2, wherein said second restraint comprises a rotation pin fixed to said body and a guide groove formed along said leg, said pin and said groove being coupled with relative sliding.

4. The teleoperated endoscopic capsule according to claim 1, wherein a first group of legs of the two separate groups is arranged near the front part of said body and a second group of legs of the two separate groups is positioned near the rear part of said body.

5. The teleoperated endoscopic capsule according to claim 1, wherein the worm screws of said driving devices are coaxial.

6. The teleoperated endoscopic capsule according to claim 1, wherein each motor is connected to the corresponding worm screw through a gearing having a transmission ratio less than 1.

7. The teleoperated endoscopic capsule according to claim 6, wherein said gearing comprises a first gearwheel fixed at the end of the drive shaft of said motor and a second gearwheel, having a diameter larger than said first gearwheel, fixed at the end of said worm screw, said two motors of said two driving devices of the two groups of legs being positioned at opposite sides of said worm screws.

8. The teleoperated endoscopic capsule according to claim 7, wherein the transmission ratio of said gearing is between 0.420 and 0.430.

9. The teleoperated endoscopic capsule according to claim 1, wherein said body is formed with longitudinal through slots on the external surface for housing said legs therein.

10. The teleoperated endoscopic capsule according to claim 9, wherein two groups of angularly equidistant slots, one for each group of legs, extend respectively from the front end and from the rear end of said body as far as an intermediate position, the slots of one group and the slots of the other group being set in two by two staggered positions.

11. The teleoperated endoscopic capsule according to claim 1, wherein the front part of said body is equipped with a transparent dome inside which the image acquisition means are mounted.

12. The teleoperated endoscopic capsule according to claim 1, wherein said at least one motor is a direct current, brushless electric motor, having an external diameter comprised between 3.5 mm and 4.5 mm and a total length comprised between 15.2 mm and 17.2 mm, said motor being equipped with a speed reducer, the maximum torque delivered to the shaft of said speed reducer being equal to approximately 2.92 mNm.

13. The teleoperated endoscopic capsule according to claim 1, wherein each group of legs is formed by a number of legs between four and six, the projections of the free ends of said legs on a plane orthogonal to the axis of said worm screws lying substantially on a same circumference whose centre coincides with said axis, said free ends being substantially equidistant from each other along said circumference, said moving means permitting the arrangement of said legs from a maximum outspread extension where said legs project outwards the exterior of said body to a minimum outspread extension where said legs are positioned along said body.

14. The teleoperated endoscopic capsule according to claim 13, wherein said legs of both groups are positioned in the maximum outspread extension, and the projections of the free ends of different groups on the plane orthogonal to the axis of the corresponding worm screws are alternated and substantially equidistant along a same circumference.

15. The teleoperated endoscopic capsule according to claim 14, wherein each leg presents, in an intermediate point between said free end and said guide groove, an elastic knee portion that provides a further degree of freedom to adapt the leg to the yielding nature of the tissue with which it makes contact.

16. The teleoperated endoscopic capsule according to claim 15, wherein two opposing extensions are provided at said knee portion for abutting with each other to limit the rotation of said leg in the a direction of its extension.

17. The teleoperated endoscopic capsule according to claim 15, wherein a further pair of extensions are provided at said knee portion for abutting with one another after a wide rotation around said knee portion.

18. The teleoperated endoscopic capsule according to claim 1, wherein the free end of each leg has a hook shape.

19. The teleoperated endoscopic capsule according to claim 1, said capsule having an overall length substantially comprised between 24 mm and 28 mm, opening angle of the legs substantially comprised between 90° and 130°, electric motors that occupy no more than 10.5% of the total volume of the body, and force generated at the free ends of each the legs substantially between 1.8N and 3.2N.

20. (canceled)

21. The teleoperated endoscopic capsule according to claim 11, wherein the image acquisition means comprise a video camera.
